(19) United States
(12) Patent Application Publication
(10) Pub. No.: US 2023/0398222 A1
(43) Pub. Date: Dec. 14, 2023

(54) METHOD FOR PRODUCING AN AMORPHOUSE SOLID DISPERSION AND PHARMACEUTICAL COMPOSITION FOR STABILIZING ACTIVE PHARMACEUTICAL INGREDIENTS

(71) Applicant: MERCK PATENT GMBH, DARMSTADT (DE)

(72) Inventors: Thomas KIPPING, Darmstadt (DE); Finn BAUER, Darmstadt (DE); Nicole DI GALLO, Darmstadt (DE); Anja-Nadine KNUETTEL, Darmstadt (DE); Alessandro Giuseppe ELIA, Darmstadt (DE)

(73) Assignee: MERCK PATENT GMBH, DARMSTADT (DE)

(21) Appl. No.: 18/034,495

(22) PCT Filed: Oct. 20, 2021
(86) PCT No.: PCT/EP2021/079788

§ 371 Date: Apr. 28, 2023

(30) Foreign Application Priority Data
Oct. 28, 2020 (EP) 20204468.1

(51) Int. Cl. A61K 47/32; A61K 9/10; A61K 9/20

(52) U.S. Cl. CPC A61K 47/32; A61K 9/10; A61K 9/2027

(57) ABSTRACT

The present invention relates to a method for producing an amorphous solid dispersion of at least one active pharmaceutical ingredient in a polymer matrix. The Invention further relates to a pharmaceutical composition using polymers as an excipient and particularly to an improved pharmaceutical composition comprising polyvinyl alcohol grades with different degrees of hydrolysis, which is suitable to stabilize active pharmaceutical ingredients.

### TECHNICAL FIELD

The present invention relates to a method for producing an amorphous solid dispersion of at least one active pharmaceutical ingredient in a polymer matrix. The Invention further relates to a pharmaceutical composition using polymers as an excipient and particularly to an improved pharmaceutical composition comprising polyvinyl alcohol grades with different degrees of hydrolysis, which is suitable to stabilize active pharmaceutical ingredients.

### STATE OF THE ART

The use of hydrophilic polymers such as polyvinyl alcohol (PVA) in a polymer matrix for pharmaceutical compositions has been widely described.

WO 2018/083285 A1 discloses powdered PVA having improved properties as a polymer matrix in pharmaceutical compositions comprising active pharmaceutical ingredients (APIs), especially in compressed tablets forming amorphous solid dispersions with poorly soluble (APIs). However, due to their high polarity, hydrophilic polymers such as almost fully hydrolyzed, i.e. 99% or 98% hydrolyzed PVAs or 88% hydrolyzed PVAs are often not able to form amorphous dispersions with poorly soluble APIs and to keep poorly soluble APIs in solution after dissolution of the matrix. Consequently, an undesirable formation of a two-phase system or recrystallization may occur thereby reducing the bioavailability of the API. Poor bioavailability is a significant problem encountered in the development of pharmaceutical compositions, particularly those containing an API that is not highly soluble. Furthermore, polymers showing high polarity tend to swell and dissolve quickly in solution thereby showing fast release rates of the API. A prolongation of the release profiles pharmaceutical matrices comprising such polymers often requires additional processing steps such as e.g. sustained release coatings. Therefore, there is a need for polymer matrices having variable properties that can be easily adapted to specific solubility characteristics and release requirements of an API.

### SUMMARY OF THE INVENTION

It was surprisingly found that a stable amorphous solid dispersion of an API in a polymer matrix comprising PVA can be obtained by mixing a first PVA having a first degree of hydrolysis, and a second PVA having a second degree of hydrolysis which is lower than the first degree of hydrolysis, with the API at an elevated temperature at which the first PVA, the second PVA and the API are in the molten state.

It was shown that even PVAs having degrees of hydrolysis differing more than 20 percentage points from each other exhibit a very good homogenous miscibility in the molten state even though the individual PVAs have highly differing solubility characteristics. The presence of PVAs having different hydrophilic/lipophilic characteristics facilitates the formation of an amorphous solid dispersion of the API in the excipient in the molten state. The presence of the more lipophilic PVA having a lower degree of hydrolysis does not only improve the formation and the stability of the amorphous solid dispersion of the API in the polymer matrix but may also improves desired sustained release properties of an oral dosage form comprising the amorphous solid dispersion.

Thus, the invention provides a method for varying and adapting the hydrophilic/lipophilic properties of a PVA polymer matrix to specific requirements regarding the solubility of the API and the desired release kinetics by combining PVAs with different hydrolysis grades.

According to the invention a “second degree of hydrolysis which is lower than the first degree of hydrolysis” refers to a difference of the hydrolysis degrees of the two PVAs, wherein the hydrolysis degree of the first PVA is at least 1 percentage point by weight higher than the second hydrolysis degree of the second PVA. Preferably, the first hydrolysis degree of the first PVA is at least 5 percentage points, 10 percentage points or 20 percentage points, points by weight higher than the second hydrolysis degree of the second PVA.

It has been found that the ratio of the first PVA and the second PVA determines the sustained release characteristics of an API in a dosage form. Therefore, according to a preferred embodiment of the invention the polymer matrix for an oral dosage form preferably comprises the first PVA and the second PVA in a weight ratio from 1:1 to 1:10. For immediate release of a poorly soluble API, preferred weight ratios of the first PVA and the second PVA are 1:2 to 1:8. For sustained release of the API preferred weight ratios of the first PVA and the second PVA are 1:1 to 1:2.

The method according to the invention is particularly suitable for obtaining an amorphous solid dispersion of an API that is poorly soluble in water. The presence of the PVA having a lower hydrolysis degree prolongs the release profile and contributes to preventing phase separation of the lipophilic API in aqueous solution. Thus, the bioavailability of the API for the patient can be enhanced.

In another aspect of the invention a pharmaceutical composition for oral administration is provided comprising an amorphous solid dispersion of at least one active pharmaceutical ingredient in a pharmaceutically acceptable polymer matrix comprising a first polyvinyl alcohol having a first degree of hydrolysis, and a second polyvinyl alcohol having a second degree of hydrolysis, wherein the amorphous solid dispersion is obtainable by a method according to the invention.

Preferred manufacturing methods for oral dosage forms including a mixing and heating step that is suitable for producing an amorphous solid dispersion of the API within the polymer matrix are hot-melt extrusion, melt extrusion, injection molding, compression molding, or additive manufacturing. These methods are commonly known processing techniques that are used in the pharmaceutical industry for the preparation of formulations comprising APIs embedded in excipients, particularly polymers.

In another aspect, the invention concerns an oral dosage form comprising the pharmaceutical composition of the invention in form of tablets, beads, granules, pellets, capsules, suspensions, emulsions, gels or films.

### DETAILED DESCRIPTION OF THE INVENTION

The present invention discloses a method for producing an amorphous solid dispersion of at least one active pharmaceutical ingredient in a polymer matrix, wherein the polymer matrix comprises polyvinyl alcohol, comprising selecting a first polyvinyl alcohol having a first degree of hydrolysis, selecting a second polyvinyl alcohol having a second degree of hydrolysis which is lower than the first degree of hydrolysis, mixing the first polyvinyl alcohol, the second polyvinyl alcohol and optionally further pharmaceutically acceptable components and the active pharmaceutical ingredient at a temperature above the glass transition temperature or melting temperature of the polymer matrix, thereby forming an amorphous solid dispersion of the active pharmaceutical ingredient. Preferably, the temperature is at least the melting temperature of the API

The amorphous solid dispersion can optionally contain further pharmaceutically acceptable components.

In another aspect, the invention discloses a pharmaceutical composition for oral administration comprising an amorphous solid dispersion of at least one API in a pharmaceutically acceptable polymer matrix comprising a first PVA having a first degree of hydrolysis, and a second PVA having a second degree of hydrolysis, wherein the amorphous solid dispersion is obtainable by using a method according to the present invention.

As used herein, the term “amorphous solid dispersion” is a dispersion of an amorphous API in a polymer matrix. Preferably, the amorphous API is distributed in a molecularly dispersed state within the polymer matrix. In this case, the solid dispersion is a solid solution. Upon dissolution, formulations comprising an amorphous solid dispersion can reach higher solubilities in aqueous media than the crystalline API.

Polyvinyl alcohol (PVA) is a synthetic water-soluble polymer that has the idealized formula [CH₂CH(OH)]n. It possesses good film-forming, adhesive, and emulsifying properties. PVA is prepared from polyvinyl acetate, where the functional acetate groups are either partially or completely hydrolysed to alcohol functional groups. If not completely hydrolysed, PVA is a random copolymer consisting of vinyl alcohol repeat units —[CH₂CH(OH)]— and vinyl acetate repeat units —[CH₂CH(OOCCH₃)]—. The polarity of PVA is closely linked to its molecular structure. The hydrolysis degree and the molecular weight determine the molecular properties of PVA. As the degree of hydrolysis of acetate groups increases, the solubility of the polymer in aqueous media and also crystallinity and melting temperature of the polymer increase. However, at high hydrolysis degrees over 88%, the solubility of PVA decreases again. PVA is generally soluble in water, but almost insoluble in almost all organic solvents, excluding, in some cases, ethanol.

The typical PVA nomenclature indicates the viscosity of a 4% solution at 20° C. and the degree of hydrolysis of the polymer. For example, PVA 4-88 is a PVA grade with a viscosity of 4 mPa s that is 88% hydrolysed, i.e. having 88% of vinyl alcohol repeat units and 12% of vinyl acetate repeat units. A skilled person is aware that a hydrolysis grade of e.g. 88% and a viscosity of 4 mPa s encompasses calculated hydrolysis grades of 87.50% to 88.49% and calculated viscosities of 3.50 mPa s to 4.49 mPa s % according to common rounding methods. Viscosity according to the invention is measured as stated in USP 39 under Monograph “Polyvinyl Alcohol” with the method Viscosity-Rotational Method (912). The degree of hydrolysis according to the invention is measured by determining the saponification value of the Polyvinyl Alcohol, e.g. as stated in USP 39 under Monograph “Polyvinyl Alcohol” under “Degree of Hydrolysis”:

Degree of Hydrolysis

Sample: 1 g of Polyvinyl Alcohol, Previously Dried at 110° to Constant Weight

Analysis:

Transfer the Sample to a wide-mouth, 250-ml conical flask fitted by means of a suitable glass joint to a reflux condenser. Add 35 ml of dilute methanol (3 in 5), and mix gently to ensure complete wetting of the solid. Add 3 drops of phenolphthalein TS, and add 0.2 N hydrochloric acid or 0.2 N sodium hydroxide if necessary, to neutralize. Add 25.0 ml of 0.2 N sodium hydroxide VS, and reflux gently on a hot plate for 1 h. Wash the condenser with 10 ml of water, collecting the washings in the flask, cool, and titrate with 0.2 N hydrochloric acid VS. Concomitantly perform a blank determination in the same manner, using the same quantity of 0.2 N sodium hydroxide VS.

Calculation of Saponification Value:

Calculate the Saponification Value:

Result=[(V_{B}−V_{S})×N×Mᵣ]/W
V_{B}=volume of 0.2 N hydrochloric acid V_{S} consumed in the titration of the blank (ml)
V_{S}=volume of 0.2 N hydrochloric acid V_{S} consumed in the titration of the Sample solution (ml)
N=actual normality of hydrochloric acid V_{S}
Mᵣ=molecular weight of potassium hydroxide, 56.11
W=weight of the portion of Polyvinyl Alcohol taken (g)

Calculation of Degree of Hydrolysis:

Calculate the Degree of Hydrolysis, Expressed as a Percentage of Hydrolysis of Polyvinyl Acetate:

Result=100−[7.84×S/(100−0.075×S))
S=saponification value of the Polyvinyl Alcohol

Preferred polymer matrices according to the present invention preferably comprise pharmaceutically acceptable PVAs having a degree of hydrolysis in the range of greater than 72.2% according to the requirements of the European Pharmacopoeia, or between 85-89% according to the United States Pharmacopoeia, and a molecular weight in the range of 14 000 g/mol to 250 000 g/mol. With increasing molecular weight, the viscosity of an aqueous solution of the PVA increases.

For the present invention, PVAs having a viscosity of 3 mPa s to 18 mPa s are preferred, PVAs having a viscosity of 3 mPa s to 10 mPa s are particularly preferred, and PVAs having a viscosity of 3 mPa s to 5 mPa s are most preferred.

Polymer matrices according to the present invention can comprise any PVA grade.

Preferred PVA grades are selected from the group consisting of PVA 15-99, PVA 28-99, PVA 2-98, PVA 3-98, PVA 4-98, PVA 5-98, PVA 6-98, PVA 10-98, PVA 15-98 PVA 20-98, PVA 30-98, PVA 30-92, PVA 3-88, PVA 4-88, PVA 5-88, PVA 6-88, PVA 8-88, PVA 13-88, PVA 18-88, PVA 23-88, PVA 26-88, PVA 32-88, PVA 40-88, PVA 3-85, PVA 4-85, PVA 5-85, PVA 3-83, PVA 4-83, PVA 5-83, PVA 3-82, PVA 4-82, PVA 5-82, PVA 3-81, PVA 4-81, PVA 5-81, PVA 3-80, PVA 4-80, PVA 5-80, PVA 26-80, PVA 32-80, PVA 15-79, PVA 3-75, PVA 3-74, PVA 3-73, PVA 3-72, PVA 4-75, PVA 4-74, PVA 4-73, PVA 4-72, PVA 5-75, PVA 5-74, PVA 5-73, PVA 5-72 or PVA 30-75.

Polymer matrices according to the present invention comprise a first PVA having a first degree of hydrolysis and second PVA having a second degree of hydrolysis which is lower than the first degree of hydrolysis. Preferably, the first hydrolysis degree of the first PVA alcohol is at least 5 percentage points by weight higher than the second hydrolysis degree of the second PVA. In one aspect, combinations of PVAs comprise a PVA having a hydrolysis degree of 88% to 99%, and a viscosity of a 4% solution at 20° C. of 2 mPas to 50 mPas, preferably 88% to 90%, and a viscosity of a 4% solution at 20° C. of 3 mPas to 40 mPas as the first PVA having a high hydrolysis degree, and a PVA having a hydrolysis degree of 70% to 83%, and a viscosity of a 4% solution at 20° C. of 2 mPas to 50 mPas, preferably 88% to 90%, and a viscosity of a 4% solution at 20° C. of 3 mPas to 40 mPas as the second PVA having a lower hydrolysis degree.

Typical combinations of PVAs comprise PVA 3-88, PVA 4-88, PVA 5-88, PVA 6-88, PVA 8-88, PVA 13-88, PVA 18-88, PVA 23-88, PVA 26-88, PVA 32-88, or PVA 40-88, as the first PVA having a high hydrolysis degree, and PVA 3-83, PVA 4-83, PVA PVA 3-82, PVA 4-82, PVA 5-82, PVA 3-81, PVA 4-81, PVA 5-81, PVA 3-80, PVA 4-80, PVA 5-80, PVA 26-80, PVA 32-80, PVA 3-79, PVA 4-79, PVA 5-79, PVA 15-79, PVA 3-75, PVA 3-74, PVA 3-73, PVA 3-72, PVA 4-75, PVA 4-74, PVA 4-73, PVA 4-72, PVA 5-75, PVA 5-74, PVA 5-73, PVA 5-72 or PVA 30-75 as the second PVA having a lower hydrolysis degree.

More preferably, the first hydrolysis degree of the first PVA alcohol is at least 10 percentage points by weight higher than the second hydrolysis degree of the second PVA.

In one aspect, combinations of PVAs comprise a PVA having a hydrolysis degree of 98% to 99%, and a viscosity of a 4% solution at 20° C. of 2 mPas to 50 mPas, preferably 98% to 99%, and a viscosity of a 4% solution at 20° C. of 2 mPas to 30 mPas as the first PVA having a high hydrolysis degree, and a PVA having a hydrolysis degree of 70% to 88%, and a viscosity of a 4% solution at 20° C. of 2 mPas to 50 mPas, preferably 72% to 88%, and a viscosity of a 4% solution at 20° C. of 3 mPas to 40 mPas as the second PVA having a lower hydrolysis degree. Typical combinations of PVAs comprise PVA 15-99, PVA 28-99, PVA 2-98, PVA 3-98, PVA 4-98, PVA 6-98, PVA 10-98, PVA 15-98, PVA 20-98, or PVA 30-98 as the first PVA having a high hydrolysis degree, and PVA 3-88, PVA 4-88, PVA 5-88, PVA 6-88, PVA 8-88, PVA 13-88, PVA 18-88, PVA 23-88, PVA 26-88, PVA 32-88, PVA 40-88, PVA 3-85, PVA 4-85, PVA 5-85, PVA 3-83, PVA 4-83, PVA 5-83, PVA 3-82, PVA 4-82, PVA 5-82, PVA 3-81, PVA 4-81, PVA 5-81, PVA 3-80, PVA 4-80, PVA 5-PVA 26-80, PVA 32-80, PVA 3-79, PVA 4-79, PVA 5-79, PVA 15-79, PVA 3-75, PVA 3-74, PVA 3-73, PVA 3-72, PVA 4-75, PVA 4-74, PVA 4-73, PVA 4-72, PVA 5-PVA 5-74, PVA 5-73, PVA 5-72 or PVA 30-75 as the second PVA having a lower hydrolysis degree.

Another typical group of combinations of PVAs comprise on one hand PVA 3-88, PVA 4-88, PVA 5-88, PVA 6-88, PVA 8-88, PVA 13-88, PVA 18-88, PVA 23-88, PVA 26-88, PVA 32-88, or PVA 40-88 as the first PVA having a high hydrolysis degree, and on the other hand PVA 3-75, PVA 4-75, PVA 5-74, PVA 30-75, PVA 3-74, PVA 4-74, PVA 5-74, PVA 3-72, PVA 4-72, or PVA 5-72 as the second PVA having a lower hydrolysis degree.

In a particularly preferred embodiment of the invention, the first hydrolysis degree of the first PVA is at least 20 percentage points by weight higher than the second hydrolysis degree of the second PVA. In one aspect, combinations of PVAs comprise a PVA having a hydrolysis degree of 98% to 99%, and a viscosity of a 4 solution at 20° C. of 2 mPas to 50 mPas, preferably 98% to 99%, and a viscosity of a 4% solution at 20° C. of 2 mPas to 30 mPas as the first PVA having a high hydrolysis degree, and a PVA having a hydrolysis degree of 70% to 75%, and a viscosity of a 4% solution at 20° C. of 2 mPas to 50 mPas, preferably 72% to 75%, and a viscosity of a 4% solution at 20° C. of 3 mPas to 30 mPas as the second PVA having a lower hydrolysis degree.

Typical combinations of PVAs comprise PVA 15-99, PVA 28-99, PVA 2-98, PVA 3-98, PVA 4-98, PVA 6-98, PVA 10-98, PVA 15-98, PVA 20-98, and PVA 30-98 as the first PVA having a high hydrolysis degree, and PVA 3-75, PVA 3-74, PVA 3-73, PVA 3-72, PVA 4-75, PVA 4-74, PVA 4-73, PVA 4-72, PVA 5-75, PVA 5-74, PVA 5-73, PVA 5-72 or PVA 30-75 as the second PVA.

In a further particularly preferred embodiment of the invention, the first hydrolysis degree of the first PVA is at least 30 or 40 percentage points by weight higher than the second hydrolysis degree of the second PVA.

The active pharmaceutical ingredients (API) in the amorphous solid dispersion according to the present invention are biologically active agents in form of a weak base, a weak acid or a neutral molecule. The API may be in the form of one or more pharmaceutically acceptable salts, esters, derivatives, analogues, prodrugs, and solvates thereof. The amorphous solid dispersion may comprise more than one API.

As used herein, the terms “poorly soluble API”, “poorly water-soluble API” and “lipophilic API” refer to an API having a solubility such that the highest therapeutic dose of the particular API to be administered to an individual cannot be dissolved in 250 ml of aqueous media ranging in pH from 1 to 8 following the definition of low solubility according to the Biopharmaceutics Classification System (BCS) classes 2 and 4. Poorly soluble APIs with weakly basic or weakly acidic characteristics have a pH-dependent solubility profile and can have a wide range of solubility in the aqueous environment of the gastrointestinal tract. APIs falling under BCS classes 2 or 4, respectively, are well known to persons skilled in the art. A typical example for a poorly soluble API of BCS class 2 is itraconazole (ITZ).

The API included in the pharmaceutical compositions of the present invention has a sufficient amount to be therapeutically effective. For a given API, therapeutically effective amounts are generally known or readily accessible by persons skilled in the art. Typically, the API may be present in the pharmaceutical composition in a weight ratio of API to the polymeric matrix the range of 1:99 to (90:10), preferably 5:95 to 60:40, most preferably 10:90 to 30:70.

It was surprisingly found that it is possible to combine a higher hydrolysis grade PVA and a lower hydrolysis grade PVA despite their different hydrophilic/lipophilic properties in the molten state resulting in the formation of a homogenous polymer matrix.

According to the invention, the polymer matrix comprising a combination of the first PVA, the second PVA and optionally further pharmaceutically acceptable components is mixed with the API at an elevated temperature. It was found that in the molten state, an API added to the molten combination of PVAs while mixing forms an amorphous solid dispersion of the API in the PVA polymer matrix at such elevated temperatures and under shear force. According to the invention, the minimum working temperature for obtaining an amorphous solid dispersion of the API is the temperature above which the polymer matrix comprising the first PVA and the second PVA are in a molten state, i.e. generally a temperature above the glass transition temperatures or melting temperatures of the first PVA and the second PVA. For facilitating the formation of a uniform distribution of the API, preferably in amorphous form, in the polymer matrix, the working temperature is preferably at least the melting temperature of the API. In case the API solubilizes in the molten polymer matrix, working temperature can also be below the melting temperature of the API.

The hydrophilic properties of the PVAs in aqueous media increase with the hydrolysis degree, however, also the crystallinity and melting point of the PVAs increase. The glass transition temperatures and melting points vary depending on the degree of hydrolysis. Fully hydrolyzed, i.e. 98-99% hydrolyzed PVAs tend to decompose at temperatures above 230° C. Therefore, typical working temperatures for obtaining an amorphous solid dispersion of an API in a PVA polymer matrix the are 140° C. to 230° C., particularly 180° C. to 200° C.

In a further embodiment of the invention, the amorphous solid dispersion may be produced in two separate melting steps. The first step being the mixing of the first PVA and the second PVA and optionally additional pharmaceutically acceptable components at a temperature above the glass transition temperature or melting temperature of the polymer mixture. After solidifying the so obtained polymer mixture is grinded. And the second step being the mixing of the solidified and grinded polymer mixture with the API at a temperature above the glass transition temperature or melting temperature of the polymer matrix, preferably at a temperature which is at least the melting temperature of the API. The API can optionally be mixed with the solidified and grinded polymer mixture before extrusion or the API can be added during extrusion of the polymer mixture.

The invention provides a method for producing a stable amorphous solid dispersion of at least one API in a polymer matrix comprising a first PVA and a second PVA having different grades of hydrolysis. This method may be employed in commonly known manufacturing methods for producing pharmaceutical compositions for oral dosage forms that include a mixing and heating step that is suitable for producing an amorphous solid dispersion of the API within a polymer matrix, and a subsequent solidifying step. Manufacturing methods employing these steps are e.g. hot-melt extrusion, melt extrusion, injection molding, compression molding, or additive manufacturing.

Furthermore, the invention provides a method for stabilizing the amorphous form of an active pharmaceutical ingredient in a polymer matrix comprising polyvinyl alcohol, said method comprising a step of mixing a first polyvinyl alcohol having a first degree of hydrolysis, a second polyvinyl alcohol having a second degree of hydrolysis which is lower than the first degree of hydrolysis and the active pharmaceutical ingredient at a temperature above the glass transition temperature or melting temperature of the polymer matrix, thereby forming an amorphous solid dispersion of the active pharmaceutical ingredient. Preferably the weight ratio of the first PVA and the second PVA is between 1:1 and 1:10, more preferably between 1:1 and 1:8. Preferably the temperature is at least the melting temperature of the active pharmaceutical ingredient.

Furthermore, the invention provides a method for stabilizing the amorphous form as described above, wherein the stability of the amorphous form of the active pharmaceutical ingredient in the amorphous solid dispersion is enhanced as compared to the stability of the amorphous form of the active pharmaceutical ingredient in the amorphous solid dispersion comprising a first PVA and a second PVA in a ratio outside the weight ratios as mentioned above.

In a further embodiment, the invention provides a use of a polymer mixture comprising a first and a second polyvinyl alcohol for stabilizing the amorphous form of an active pharmaceutical ingredient in an amorphous solid dispersion by mixing the first polyvinyl alcohol having a first degree of hydrolysis, the second polyvinyl alcohol having a second degree of hydrolysis which is lower than the first degree of hydrolysis and the active pharmaceutical ingredient at a temperature above the glass transition temperature or melting temperature of the polymer matrix, thereby forming an amorphous solid dispersion of the active pharmaceutical ingredient.

Preferably the weight ratio of the first PVA and the second PVA is between 1:1 and 1:10, more preferably between 1:1 and 1:8, most preferably between 1:3, 5 and 1:8. Preferably the temperature is at least the melting temperature of the active pharmaceutical ingredient.

Furthermore, it was surprisingly found that a tailored release of more than 80% of API within 90 minutes dissolution time can be achieved by varying the weight ratios of the first PVA with a first degree of hydrolysis to a second PVA with a second degree of hydrolysis from 1:1 to 1:8, preferably between 1:3, 5 to 1:8, contrary to the samples with a weight ratios of from 8:1 to 1:1, as explained in Example 3 and depicted in FIG. 3. Preferably, the first hydrolysis degree of the first polyvinyl alcohol is at least 5 percentage points by weight higher than the second hydrolysis degree of the second polyvinyl alcohol.

All preferred embodiments mentioned above for the method of producing the amorphous solid dispersion are also preferred for the method for stabilizing and the use of a polymer mixture, including but not limited to the preferred PVA grades of the first and a second polyvinyl alcohol, the API or the weight ratios of the first PVA and the second PVA.

The presence of different grade PVAs allows producing a homogenous stable amorphous solid dispersion. In case of low soluble API such as itraconazole the presence of a low hydrolyzed PVA has a stabilizing effect on the amorphous state. The use of further stabilizing processing agents such as plasticizers or thermal lubricants can be avoided. Therefore, such additives cannot influence the drug release characteristics of a resulting pharmaceutical composition.

X-ray diffraction analysis revealed that in ternary matrices having a weight ratio of the first PVA (higher degree of hydrolysis) and the second PVA (lower degree of hydrolysis) from 1:1 to 1:10 an amorphous solid dispersion of a poorly soluble API can be obtained which is substantially free of detectable crystalline material. The absence of crystalline API in the polymer matrix is highly desirable for a high absorption of the API in vivo.

It was shown by dissolution experiments that the combination of a PVA grades with different degrees of hydrolysis in the polymer matrix may have a desirable effect on sustained release properties of the API. It was found that weight ratios of the first PVA (higher degree of hydrolysis) and the second PVA (lower degree of hydrolysis) in a ratio between 1:1 and 1:3.5, preferably between 1:1 and 1:2, cause retardation of the release of the API.

Sustained release oral dosage forms release the API from the dosage form in a pre-determined controlled manner, thereby continuously administering the API to the body and providing a therapeutically effective blood level of the API over an extended period of time. The advantages of such retarded pharmaceutical compositions are the avoidance of unwanted possibly toxic plasma levels of the API and a reduction in the frequency of administration of the dosage form resulting in an improvement of the patient compliance.

Therefore, the use of different grade PVAs in different ratios in a polymer matrix is of particular interest for the formulation of solid oral pharmaceutical dosage forms with a prolonged API release such that the API is released evenly over a prolonged period of time. It is assumed that such oral dosage forms do not dissolve directly in aqueous solution, such as in the mouth or gastrointestinal tract, but swell and the drug is released by diffusion only gradually. By varying the ratio of a first PVA with high grade of hydrolysis and a second PVA with a lower grade of hydrolysis in the polymer matrix, the chemical properties of the polymer matrices may be tuned to achieve a range of more or less sustained API release characteristics depending on the desired administration form.

Dissolution experiments further revealed that in a polymer matrix comprising an amorphous solid solution of the API, certain weight ratios of highly hydrolyzed PVA and a low hydrolyzed PVA may support the supersaturated solubility of the API in aqueous solution. Preferred weight ratios of highly hydrolyzed PVA and a low hydrolyzed PVA for obtaining a rapid and substantially full release of a poorly soluble API are in the range of 1:2 to 1:8. It is assumed that the presence of the mixture of a more lipophilic lower grade PVA and a hydrophilic higher grade PVA in aqueous solution prevents crystallization and phase separation of a poorly water-soluble API in aqueous media. Since a low water solubility of an API in general accompanies a low bioavailability after its administration in a pharmaceutical preparation, the pharmaceutical compositions according to the invention also contribute to improving the bioavailability of poorly water-soluble APIs.

As used herein, “bioavailability” is a term meaning the degree to which a drug becomes available to the target tissue after being administered to the body of a patient.

Combining different hydrolysis grade PVAs in a polymer matrix according to the method of the present invention allows formulation developers to fine-tune specific properties of the resulting pharmaceutical composition. Particularly, the release profile of a certain API can be adapted to solubility characteristics and the desired dosage mode of the targeted API by selecting and combining appropriate PVA grades and thereby varying the polarity of the polymer matrix.

It was found that increasing percentages of lower grade PVA, such as PVA 5-74, support the solubilization of lipophilic APIs in the PVA polymer matrix and stabilize the amorphous solid dispersion, whereas higher grade PVA, such as PVA 4-98, assures the release of the API into aqueous media.

The polymer matrix comprising different hydrolysis grade PVAs may be combined with other pharmaceutically acceptable excipients. Particularly, the pharmaceutical composition according to the invention may comprise additional pharmaceutically acceptable hydrophilic or lipophilic polymers. The pharmaceutical composition may also comprise pharmaceutically acceptable fillers, plasticizers, surfactants, and other suitable components that are well known to those skilled in the art.

As used herein, the phrase “pharmaceutically acceptable” refers to all compounds, such as solvents, dispersion media, excipients, carriers, coatings, active agents, isotonic and absorption delaying agents, and the like that do not produce an allergic or similar untoward reaction when administered to humans in general. The use of such media and agents in pharmaceutical compositions is well known in the art.

### BRIEF DESCRIPTION OF THE FIGURES

FIG. 1 shows a table summarizing extrusion parameters for preparing model ternary matrix systems with varying ratios of PVA 5-74 and PVA 4-98 and itraconazole (ITZ) as a lipophilic model API.

FIG. 2 shows X-ray diffractograms of extruded matrices with varying ratios of PVA and PVA 4-98 and a constant load of the model API itraconazole (ITZ) at 10% by weight.

FIG. 3 shows dissolution profiles of ternary systems containing PVA 4-98, PVA 5-74 and ITZ.

### EXAMPLES

### Example 1: Preparation of Ternary Compositions

Five ternary compositions comprising different ratios of PVA 4-98 (Poval 4-98, Kuraray Europe GmbH) and PVA 5-74 (Poval 5-74, Kuraray Europe GmbH) and to comparative compositions comprising solely PVA 4-98 or PVA 5-74 as the polymer matrix were prepared by hot-melt extrusion with 10% by weight itraconazole (ITZ) according to Table 1 as follows:

TABLE 1
Ternary compositions

| PVA 4-98:PVA 5-74:API | PVA 4-98:PVA 5-74 |
|---|---|
| 10:80:10 | 1:8 |
| 20:70:10 | 1:3.5 |
| 45:45:10 | 1:1 |
| 70:20:10 | 3.5:1 |
| 80:10:10 | 8:1 |
| 0:90:10 | 0:1 |
| 90:0:10 | 1:0 |

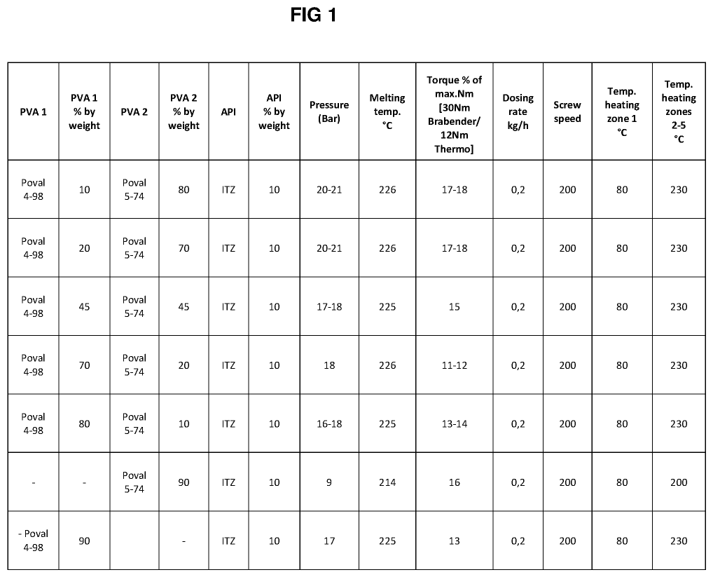

The quantities of the first polyvinyl alcohol Poval 4-98, the second polyvinyl alcohol Poval 5-74 and the active ingredient itraconazole (ITZ) required for a total mass of 200 g powder mixture according to the weight ratios shown in Table 1 and FIG. 1 were weighed into a 1 L mixing vessel and then mixed by means of a tubular mixer for 5 min.

The powder mixture was then filled into the gravimetric twin-screw feeder of a Brabender KETSE 12/36 extruder and a determination of the maximum feed rate was performed.

The heating zones were heated at the respective target temperatures as shown in FIG. 1.

After the heating zones had reached their respective temperatures, the speed and, analogously, the dosing rate of the powder mixture was increased step by step in units of 50 until the target speed and target dosing rate of 200 rpm and 200.0 g/h, respectively, were reached. The extrudate was discarded for about 5 minutes until nozzle pressure and torque stabilized. The extrudate was then allowed to cool on the conveyor belt at room temperature and thereby conveyed to the pelletizer, where the extrudate was crushed to 1.5 mm pellets using a Brabender pelletizer. The process was continued until the powder mixture in the feeder was used up. This was reflected in incipient fluctuations in the dosing rate. Afterwards, the dosing was stopped and the screw speed was gradually reduced to 10 rpm and held for another 10 minutes to feed residual polymer from the extruder barrel, which was then discarded.

The so obtained extruded ternary composition pellets were used for further x-ray diffractometry analysis and dissolution experiments.

### Example 2: X-Ray Diffractometry Analysis (XRD) of Ternary Compositions

The extruded polymer matrices obtained according to Example 1 were further characterized by X-ray diffractometry analysis. X-ray diffraction is a well-established technology in pharmaceutical sciences that can be used to identify the polymorphic form of the API as well as the remaining crystallinity of a polymer.

Samples were measured in transmission mode at 40 KV and 40 mA. Copper was used as an anode material at a wavelength of 1.54060 A. The stepsize was 0.015 at 15.0 sec/step.

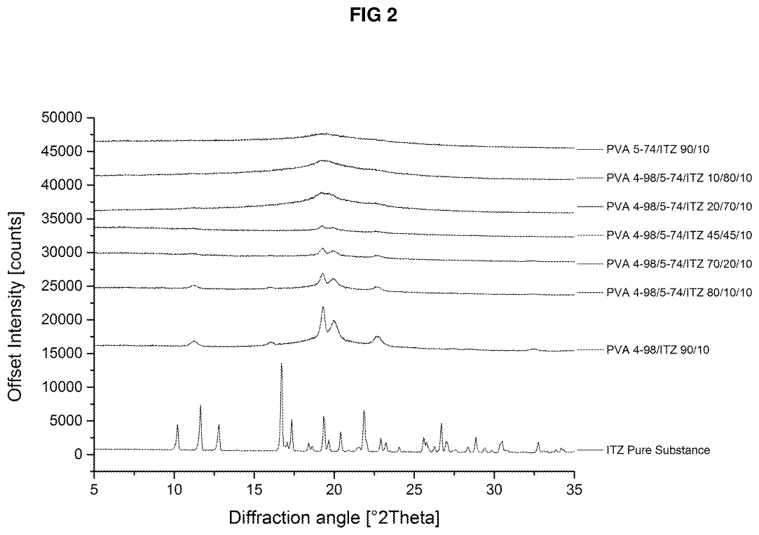

The stabilizing effect of the low hydrolysed PVA 5-74 on the amorphous state of ITZ was evaluated. The X-ray diffractograms of the extruded matrices are shown in FIG. 2. It was observed that in a pure PVA 4-98 based polymer matrix, crystalline peaks of ITZ were still detected. With an increasing percentage of PVA 5-74 corresponding to a weight ratio of PVA 4-98 to PVA 5-74 from 8:1 to 1:8 in the polymer matrix, the crystalline peaks were more and more reduced and showed only two minor peaks for a ternary composition having a weight ratio of PVA 4-98 to PVA 5-74 of 1:1. Ternary compositions having a weight ratio of PVA 4-98 to PVA 5-74 from 1:3, 5 to 1:8 showed no distinct crystalline peaks. From the results, it can be confirmed hat an increasing amount of the acetate groups induced by an increasing percentage of the lower hydrolysed PVA 5-74 in the polymer matrix improves the stability of the amorphous state of ITZ. Thus, the addition of PVA 5-74 in a weight ratio of PVA 4-98 to PVA 5-74 from 1:1 to 1:8 was sufficient to effectively reduce the crystalline content of the polymer matrix indicating a successful stabilization of the amorphous ITZ within the polymer matrix.

### Example 3: Drug Release of Ternary Compositions

Drug release experiments were performed using the extruded polymer matrices obtained according to Example 1.

### Sample Preparation

The ternary composition extrudates were ground in an IKA Tubemill 100 with a 40 ml disposable grinding cup for 20 sec at 25000 rpm. 3 samples of each extrudate were prepared. For each sample, 500 mg of extrudate were weighed corresponding to 50 mg ITZ per sample.

Dissolution Method:

The dissolution rates of ITZ from the ternary composition extrudates were measured using a Sotax AT7 smart measuring system equipped with an online Agilent photometer 8453. The samples were placed in dissolution vessels containing 900 mL SGF.sp (20 g NaCl, 800 mL 0.1 M HCl ad 10.0 L deionized water) equilibrated to a temperature of 37±0.5° C. with a paddle rotation of 75 rpm. Samples were taken at 5, 20, 35, 50, 65, 95, 125, 155, 185 and 240 min, filtered and analyzed by HPLC.

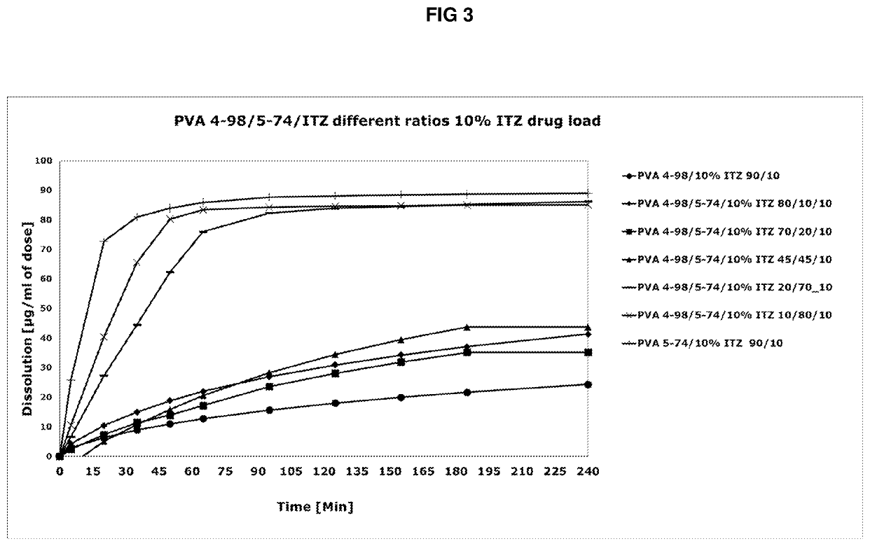

The dissolution profiles of the ternary compositions are shown in FIG. 3. Dissolution data show that the release profiles of ternary compositions having weight ratios of PVA 4-98 to PVA 5-74 of 1:3.5 to 1:8 were very similar showing more than 80% release of ITZ within 90 min dissolution time.

Therefore, varying the weight ratios of PVA 4-98 to PVA 5-74 and enables a tailored release formulation.

1. A method for producing an amorphous solid dispersion of at least one active pharmaceutical ingredient in a polymer matrix, wherein the polymer matrix comprises polyvinyl alcohol, comprising selecting a first polyvinyl alcohol having a first degree of hydrolysis, selecting a second polyvinyl alcohol having a second degree of hydrolysis which is lower than the first degree of hydrolysis, mixing the first polyvinyl alcohol, the second polyvinyl alcohol and the active pharmaceutical ingredient at a temperature above the glass transition temperature or melting temperature of the polymer matrix, thereby forming an amorphous solid dispersion of the active pharmaceutical ingredient.

2. The method according to claim 1, wherein the degree of hydrolysis is measured by determining the saponification value of the polyvinyl alcohol.

3. The method according to claim 1, wherein the temperature is at least the melting temperature of the active pharmaceutical ingredient.

4. The method according to claim 1, wherein the amorphous solid dispersion is produced by hot-melt extrusion, melt extrusion, injection molding, compression molding, or additive manufacturing.

5. The method according to claim 1, wherein the first hydrolysis degree of the first polyvinyl alcohol is at least 5 percentage points by weight higher than the second hydrolysis degree of the second polyvinyl alcohol.

6. The method according to claim 1, wherein the first polyvinyl alcohol is a PVA having a hydrolysis degree of 98% to 99%, and a viscosity of a 4% solution at 20° C. of 2 mPas to 50 mPas, and the second polyvinyl alcohol is a PVA having a hydrolysis degree of 70% to 88%, and a viscosity of a 4% solution at 20° C. of 2 mPas to 50 mPas.

7. The method according to claim 1, wherein the weight ratio of the first PVA and the second PVA is from 1:1 to 1:8.

8. The method according to claim 1, wherein the weight ratio of the first PVA and the second PVA is from 1:3.5 to 1:8.

9. The method according to claim 1, wherein the active pharmaceutical ingredient is poorly soluble in water.

10. A pharmaceutical composition for oral administration comprising an amorphous solid dispersion of at least one active pharmaceutical ingredient in a pharmaceutically acceptable polymer matrix comprising a first polyvinyl alcohol having a first degree of hydrolysis, and a second polyvinyl alcohol having a second degree of hydrolysis, wherein the amorphous solid dispersion is obtainable by a method according to claim 1.

11. An oral dosage form comprising a pharmaceutical composition according to claim 10 in form of tablets, beads, granules, pellets, capsules, suspensions, emulsions, gels or films.

12. A method for stabilizing the amorphous form of an active pharmaceutical ingredient in a polymer matrix comprising polyvinyl alcohol, said method comprising a step of mixing a first polyvinyl alcohol having a first degree of hydrolysis, a second polyvinyl alcohol having a second degree of hydrolysis which is lower than the first degree of hydrolysis and the active pharmaceutical ingredient at a temperature above the glass transition temperature or melting temperature of the polymer matrix, thereby forming an amorphous solid dispersion of the active pharmaceutical ingredient, whereas the weight ratio of the first PVA and the second PVA is between 1:1 and 1:10.

13. The method according to claim 12, wherein the temperature is at least the melting temperature of the active pharmaceutical ingredient.

14. The method according to claim 12, wherein the stability of the amorphous form of the active pharmaceutical ingredient in the amorphous solid dispersion is enhanced as compared to the stability of the amorphous form of the active pharmaceutical ingredient in the amorphous solid dispersion comprising a first PVA and a second PVA in a ratio outside the weight ratio between 1:1 and 1:10.

15. A method for stabilizing the amorphous form of an active pharmaceutical ingredient in an amorphous solid dispersion comprising
mixing a first polyvinyl alcohol having a first degree of hydrolysis, a second polyvinyl alcohol having a second degree of hydrolysis which is lower than the first degree of hydrolysis and an active pharmaceutical ingredient at a temperature above the glass transition temperature or melting temperature of the polymer matrix, thereby forming an amorphous solid dispersion of the active pharmaceutical ingredient,
wherein the weight ratio of the first PVA and the second PVA is between 1:1 and 1:10.
